(19) United States
(12) Patent Application Publication
(10) Pub. No.: US 2026/0174716 A1
(43) Pub. Date: Jun. 25, 2026

(54) CRYSTAL FORM OF ALKYL CARBOXYLIC ACID COMPOUND AND USE THEREOF

(71) Applicants: CONSUN PHARMACEUTICAL (INNER MONGOLIA) CO. LTD, Inner Mongolia (CN); MEDSHINE DISCOVERY INC., Jiangsu (CN)

(72) Inventors: Yunfu LUO, Shanghai (CN); Weizhi GE, Shanghai (CN); Sheng SU, Shanghai (CN); Guoli ZHANG, Shanghai (CN)

(21) Appl. No.: 19/127,420

(22) PCT Filed: Nov. 8, 2023
(86) PCT No.: PCT/CN2023/130485

§ 371 Date: May 6, 2025

(30) Foreign Application Priority Data
Nov. 8, 2022 (CN) 202211394422.2

(51) Int. Cl. A61K 31/196; C07C 233/54

(52) U.S. Cl. CPC A61K 31/196; C07C 233/54

(57) ABSTRACT

Disclosed in the present invention are a crystal form of an alkyl carboxylic acid compound and the use thereof. Specifically disclosed are a crystal form of the compound as represented by formula (I) and the use thereof.

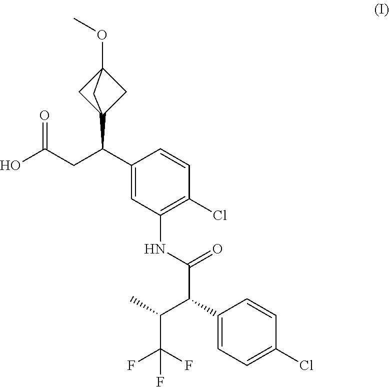 [SMILES: COC12CC([C@H](CC(=O)O)c3ccc(Cl)c(NC(=O)[C@H](c4ccc(Cl)cc4)[C@@H](C)C(F)(F)F)c3)(C1)C2]

The present disclosure claims the right of the following priority:

Application No.: CN202211394422.2, application date: Nov. 8, 2022.

### TECHNICAL FIELD

The present disclosure relates to a crystal form of an alkyl carboxylic acid compound and a use thereof, and specifically relates to a crystal form of a compound of formula (I) and a use thereof.

### BACKGROUND

Soluble guanylate cyclase (sGC) is an enzyme-linked receptor for the second messenger nitric oxide (NO) and is widely present in various cell types, including muscle, epithelial, neuronal, and endothelial cells. sGC is a heterodimer consisting of either an al or α2 subunit bound to a β1 subunit. The β1 subunit contains a heme cofactor and serves as the key signal transduction enzyme in the NO-sGC-cGMP signaling pathway. Under physiological conditions, NO binds to the heme cofactor of sGC, and upon activation, it catalyzes the conversion of guanosine-5′-triphosphate (GTP) to cyclic guanosine monophosphate (cGMP).

cGMP is a significant secondary messenger molecule. By activating a variety of downstream effector molecules, such as phosphodiesterase (PDE), cyclic nucleotide-gated ion channel (CNG), and protein kinase (PKG), it subsequently triggers a series of downstream cascade reactions and plays critical physiological roles in the gastrointestinal system, circulatory system, and nervous system. Functions include promoting vasodilation and smooth muscle relaxation, inhibiting platelet aggregation, vascular remodeling, apoptosis, and inflammation, as well as participating in neurotransmission. Therefore, sGC stimulators can serve as potential therapeutic agents for treating cardiovascular diseases (heart failure, pulmonary arterial hypertension, angina, myocardial infarction) and fibrotic diseases (renal fibrosis, systemic sclerosis). Under the aforementioned pathological conditions, prolonged oxidative stress can lead to the oxidation of the heme cofactor in sGC (from a ferrous state to a ferric state), which causes the sGC enzyme unresponsive to NO activation and may exacerbate the progression of the disease. It further contributes to diseases such as endothelial dysfunction, atherosclerosis, hypertension, stable or unstable angina, thrombosis, myocardial infarction, stroke, or worsening of erectile dysfunction. Therefore, activating the oxidized sGC to produce cGMP makes the treatment and/or prevention of such diseases possible.

sGC activators are independent of NO and also independent of the heme cofactor, and are capable of directly activating the sGC-cGMP signaling pathway. sGC activators have the potential to offer benefits in many diseases caused by defective NO signaling pathway, particularly following oxidative stress.

Addressing the current market and clinical unmet needs for such soluble guanylate cyclase stimulators, the present disclosure provides a crystal form of a new class of compounds as well as a preparation method therefor. These compounds can act as activators of soluble guanylate cyclase, exhibiting excellent in vitro stimulatory activity on soluble guanylate cyclase and favorable pharmacokinetic properties.

### CONTENT OF THE PRESENT INVENTION

The present disclosure provides a crystal form A of a compound of formula (I), wherein the crystal form A of the compound of formula (I) has an X-ray powder diffraction pattern comprising characteristic diffraction peaks at the following 2θ angles: 15.540±0.200°, 16.100±0.200°, and 17.601±0.200°,

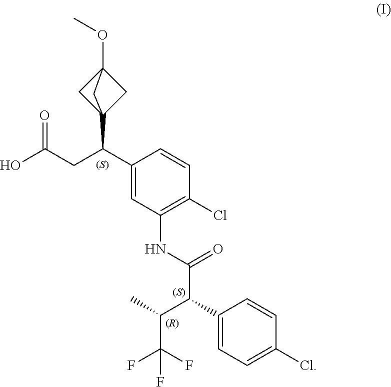

In some embodiments of the present disclosure, the X-ray powder diffraction pattern for the crystal form A of the compound of formula (I) comprises characteristic diffraction peaks at the following 2θ angles: 11.041±0.200°, 14.381±0.200°, 15.540±0.200°, 16.100±0.200°, 17.601±0.200°, 18.281±0.200°, 18.799±0.200°, and 22.903±0.200°.

In some embodiments of the present disclosure, the X-ray powder diffraction pattern for the crystal form A of the compound of formula (I) comprises characteristic diffraction peaks at the following 2θ angles: 3.441±0.200°, 8.113±0.200°, 8.799±0.200°, 11.041±0.200°, 14.381±0.200°, 15.540±0.200°, 16.100±0.200°, 17.601±0.200°, 18.281±0.200°, 18.799±0.200°, 22.903±0.200°, and 23.682±0.200°.

In some embodiments of the present disclosure, the X-ray powder diffraction pattern for the crystal form A of the compound of formula (I) comprises characteristic diffraction peaks at the following 2θ angles: 3.441±0.200°, 7.198±0.200°, 8.113±0.200°, 8.799±0.200°, 11.041±0.200°, 13.863±0.200°, 14.381±0.200°, 15.540±0.200°, 16.100±0.200°, 17.601±0.200°, 18.281±0.200°, 18.799±0.200°, 19.523±0.200°, 22.903±0.200°, 23.682±0.200°, and 24.940±0.200°.

In some embodiments of the present disclosure, the X-ray powder diffraction pattern for the crystal form A of the compound of formula (I) comprises characteristic diffraction peaks at the following 2θ angles: 3.441±0.200°, and/or 7.198±0.200°, and/or 8.113±0.200°, and/or 8.799±0.200°, and/or 9.914±0.200°, and/or 11.041±0.200°, and/or 11.880±0.200°, and/or 13.863±0.200°, and/or 14.381±0.200°, and/or 15.540±0.200°, and/or 16.100±0.200°, and/or 17.063±0.200°, and/or 17.601±0.200°, and/or 18.281±0.200°, and/or 18.799±0.200°, and/or 19.523±0.200°, and/or 20.333±0.200°, and/or 21.164±0.200°, and/or 21.761±0.200°, and/or 22.176±0.200°, and/or 22.903±0.200°, and/or 23.682±0.200°, and/or 24.940±0.200°, and/or 25.8230.200°, and/or 26.539±0.200°, and/or 28.124±0.200°, and/or 28.756±0.200°, and/or 30.261±0.200°, and/or 31.477±0.200°, and/or 32.582±0.200°, and/or 36.303±0.200°, and/or 38.141±0.200°, and/or 38.742±0.200°.

In some embodiments of the present disclosure, the crystal form A of the compound of formula (I) has an XRPD pattern as shown in FIG. 1.

In some embodiments of the present disclosure, analysis data of the XRPD pattern for the crystal form A of the compound of formula (I) are as shown in Table 1.

TABLE 1
Analysis data of XRPD pattern of
crystal form A of compound of formula (I)

| | | | | Peak |
|---|---|---|---|---|
| | 2θ angle | d-Spacing | Peak | height |
| No. | (°) | (Å) | height | (%) |
| 1 | 3.441 | 25.6736 | 301 | 23.9 |
| 2 | 7.198 | 12.2804 | 207 | 16.5 |
| 3 | 8.113 | 10.8982 | 385 | 30.6 |
| 4 | 8.799 | 10.0498 | 387 | 30.7 |
| 5 | 9.914 | 8.9214 | 190 | 15.1 |
| 6 | 11.041 | 8.0137 | 624 | 49.5 |
| 7 | 11.880 | 7.4492 | 88 | 7.0 |
| 8 | 13.863 | 6.3881 | 282 | 22.4 |
| 9 | 14.381 | 6.1590 | 618 | 49.1 |
| 10 | 15.540 | 5.7022 | 1000 | 79.4 |
| 11 | 16.100 | 5.5053 | 1026 | 81.4 |
| 12 | 17.063 | 5.1967 | 176 | 14.0 |
| 13 | 17.601 | 5.0388 | 1259 | 100.0 |
| 14 | 18.281 | 4.8530 | 392 | 31.2 |
| 15 | 18.799 | 4.7205 | 794 | 63.1 |
| 16 | 19.523 | 4.5469 | 301 | 23.9 |
| 17 | 20.333 | 4.3676 | 82 | 6.5 |
| 18 | 21.164 | 4.1980 | 51 | 4.1 |
| 19 | 21.761 | 4.0840 | 119 | 9.4 |
| 20 | 22.176 | 4.0086 | 128 | 10.2 |
| 21 | 22.903 | 3.8830 | 399 | 31.7 |
| 22 | 23.682 | 3.7569 | 311 | 24.7 |
| 23 | 24.940 | 3.5703 | 227 | 18.0 |
| 24 | 25.823 | 3.4501 | 62 | 4.9 |
| 25 | 26.539 | 3.3586 | 165 | 13.1 |
| 26 | 28.124 | 3.1728 | 46 | 3.6 |
| 27 | 28.756 | 3.1046 | 164 | 13.0 |
| 28 | 30.261 | 2.9535 | 67 | 5.3 |
| 29 | 31.477 | 2.8421 | 53 | 4.2 |
| 30 | 32.582 | 2.7483 | 104 | 8.2 |
| 31 | 36.303 | 2.4746 | 60 | 4.8 |
| 32 | 38.141 | 2.3595 | 100 | 8.0 |
| 33 | 38.742 | 2.3242 | 48 | 3.9 |

In some embodiments of the present disclosure, the crystal form A of the compound of formula (I) has a differential scanning calorimetry (DSC) curve comprising endothermic peaks with an onset at 164.69° C.±5.00° C.

In some embodiments of the present disclosure, the crystal form A of the compound of formula (I) has a DSC pattern as shown in FIG. 2.

In some embodiments of the present disclosure, the crystal form A of the compound of formula (I) has a thermogravimetric analysis (TGA) curve with no weight loss before the melting point.

In some embodiments of the present disclosure, the crystal form A of the compound of formula (I) has a TGA pattern as shown in FIG. 3.

The present disclosure further provides a use of the crystal form A of the compound of formula (I) in the manufacture of a medicament for treating chronic kidney disease.

### Technical Effect

The compounds of the present disclosure exhibit significant in vitro stimulatory activity on guanylate cyclase and possess excellent pharmacokinetic properties. The crystal form of the compound according to the present disclosure is stable, non-hygroscopic or virtually non-hygroscopic, and less susceptible to light and heat.

### Definition and Description

Unless otherwise specified, the following terms and phrases when used herein have the following meanings. A specific phrase or term should not be considered indefinite or unclear in the absence of a particular definition, but should be understood in the ordinary sense. When a trading name appears herein, it is intended to refer to its corresponding commodity or active ingredient thereof.

The intermediate compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include, but are not limited to, the examples of the present disclosure.

The chemical reactions of the specific embodiments of the present disclosure are completed in a suitable solvent, and the solvent must be appropriate for the chemical changes of the present disclosure and the required reagents and materials thereof. To obtain the compounds of the present disclosure, it is sometimes necessary for those skilled in the art to modify or select the synthetic steps or reaction processes based on the existing embodiments.

For any given crystal form, the relative intensities of diffraction peaks may change due to preferred orientation caused by factors such as crystal morphology, which is well-known in the field of crystallography. Where the influence of preferred orientation exists, the peak intensities are altered, but the diffraction peak positions of the crystal form cannot be changed. Furthermore, for any given crystal form, there may be slight errors in peak positions, which is also well-known in the field of crystallography. For example, due to variations in temperature during sample analysis, sample displacement, or instrument calibration, the peak positions may shift, and the measurement error for 2θ values is sometimes approximately ±0.2 degrees. Therefore, it is well-known to those skilled in the art that this error should be taken into account when determining each crystal structure.

DSC measures the transition temperature at which a crystal absorbs or releases heat due to changes in its crystal structure or crystal melting. For the same crystal form of the same compound, in continuous analyses, the thermal transition temperature and melting point typically exhibit errors within approximately 5° C. or 3° C. When we state that a compound has a given DSC peak or melting point, this refers to the DSC peak or melting point with ±5° C. or ±3° C. deviations. DSC provides an auxiliary method for distinguishing different crystal forms. Different crystal forms can be identified based on their distinct transition temperature characteristics. It should be noted that for mixtures, their DSC peaks or melting points may vary over a wider range. Furthermore, since decomposition accompanies the melting process of the substance, the melting temperature is associated with the heating rate.

For the same crystal form, the temperature at which weight loss occurs in TGA may vary due to factors such as the measuring instrument, measurement method/conditions, etc. For any specific crystal form, there may be an error in the temperature at which weight loss occurs, and the error may be approximately ±5° C. or approximately ±3° C.

It should be noted that, as drug molecules come in contact with solvent molecules during the preparation of drug crystal forms, it is difficult to avoid situations where external conditions and internal factors cause solvent molecules and compound molecules to form co-crystals and remain in the solid substance, thereby forming solvates. These specifically include stoichiometric solvates and non-stoichiometric solvates. Such solvates are encompassed within the scope of the present disclosure.

The structure of the compounds of the present disclosure can be confirmed by conventional methods known to those skilled in the art, and if the present disclosure involves an absolute configuration of a compound, then the absolute configuration can be confirmed by means of conventional techniques in the art. For example, in the case of single crystal X-ray diffraction (SXRD), diffraction intensity data are collected from the cultured single crystal using a Bruker D8 venture diffractometer with CuKα radiation as the light source and scanning mode: φ/ω scan, and after collecting the relevant data, the crystal structure is further analyzed by direct method (Shelxs97), so that the absolute configuration can be confirmed.

Unless otherwise specified, in the DSC pattern, the upward peak is exothermic.

The present disclosure is described in detail by the examples below, but these examples do not imply any restrictions on the present disclosure.

All solvents used in the present disclosure are commercially available and can be used without further purification.

The compounds of the present disclosure are named according to the conventional naming principles in the art or by ChemDraw® software, and the commercially available compounds use the supplier catalog names.

### X-Ray Powder Diffraction (X-Ray Powder Diffractometer, XRPD) Method of the Present Disclosure

Instrument model: D2 Phaser X-ray diffractometer
Test method: Approximately 3 to 10 mg of sample is used for XRPD detection.
The detailed XRPD parameters are as follows:
Light tube: Cu, kα, (λ=1.5418 Å).
Light tube voltage: 30 kV, light tube current: 10 mA
Divergence slit: 0.6 mm
Detector slit: 0.075 mm
Anti-scatter slit: 1 mm
Scan range: 3 to 40 deg
Step size: 0.02 deg
Scan time: 0.2 s

### Differential Scanning Calorimetry (Differential Scanning Calorimeter, DSC) Method of the Present Disclosure

Instrument model: TA DSC 250 differential scanning calorimeter

A sample (1 to 5 mg) is placed in an alumina crucible with cover lid and tested under the protection of 50 mL/min dry nitrogen, and the method is: heating from 25° C. to the set test temperature at a rate of 10° C./min.

### Thermogravimetric Analysis (Thermal Gravimetric Analyzer, TGA) Method of the Present Disclosure

Instrument model: TA TGA 550 thermal gravimetric analyzer
Test method: A sample (2 to 5 mg) is placed in an uncovered aluminum crucible and tested under the protection of 60 mL/min dry nitrogen, and the method is: heating from room temperature to 300° C. at a rate of 10° C./min.

### Dynamic Vapor Sorption (DVS) Method of the Present Disclosure

Instrument model: SMS (Intrinsic Plus) Dynamic Vapor Sorption Analyzer
Test conditions: The sample (30 to 50 mg) is taken and placed in a DVS sample dish for testing.

The detailed DVS parameters are as follows:
Temperature: 25° C.
Equilibrium: dm/dt<0.002%/min (equilibrium time: 1 h)
Drying: Dry at 0% RH for 120 min
Humidity cycle: 0%-95%-0% RH
Gradient: 0-90% RH, 10% RH; 90%-95% RH, 5% RH
RH (%) test gradient range: 0%-95%

The evaluation and classification of hygroscopicity are shown in Table 2:

TABLE 2
Evaluation and classification of hygroscopicity

| Classification of hygroscopicity | ΔW % |
|---|---|
| Deliquescence | Absorption of sufficient |
| | water to form a liquid |
| Highly hygroscopic | ΔW % ≥ 15% |
| Hygroscopic | 15% > ΔW % ≥ 2% |
| Slightly hygroscopic | 2% > ΔW % ≥ 0.2% |
| Non-hygroscopic or virtually | ΔW % < 0.2% |
| non-hygroscopic | |

Note:
ΔW % represents the hygroscopic weight gain of the test sample at 25 ± 1° C. and 80 ± 2% RH.

### BRIEF DESCRIPTION OF THE DRAWINGS

FIG. 1 shows the XRPD pattern for the crystal form A of the compound of formula (I) under Cu-Kα radiation.

FIG. 2 shows the DSC pattern for the crystal form A of the compound of formula (I).

FIG. 3 shows the TGA pattern for the crystal form A of the compound of formula (I).

FIG. 4 shows the DVS pattern for the crystal form A of the compound of formula (I).

FIG. 5 shows a molecular structure diagram of the compound of formula (I).

FIG. 6 shows an ellipsoid diagram of the molecular structure of the compound of formula (I).

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and its specific examples have also been disclosed; for those skilled in the art, it is obvious to make various modifications and improvements to the specific examples of the present disclosure without departing from the spirit and scope of the present disclosure.

### Example 1: Preparation of Compound of Formula (I)

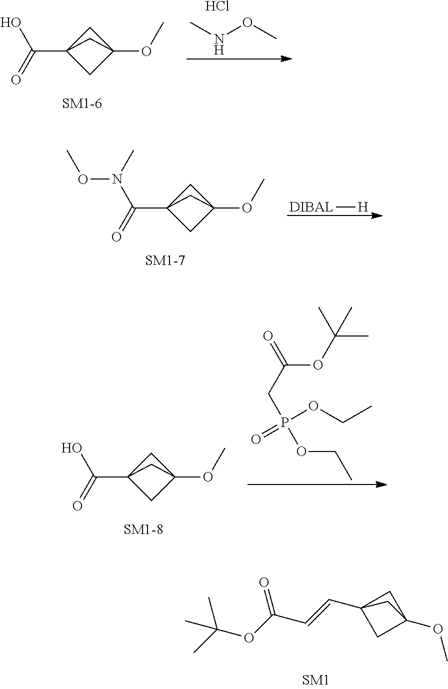

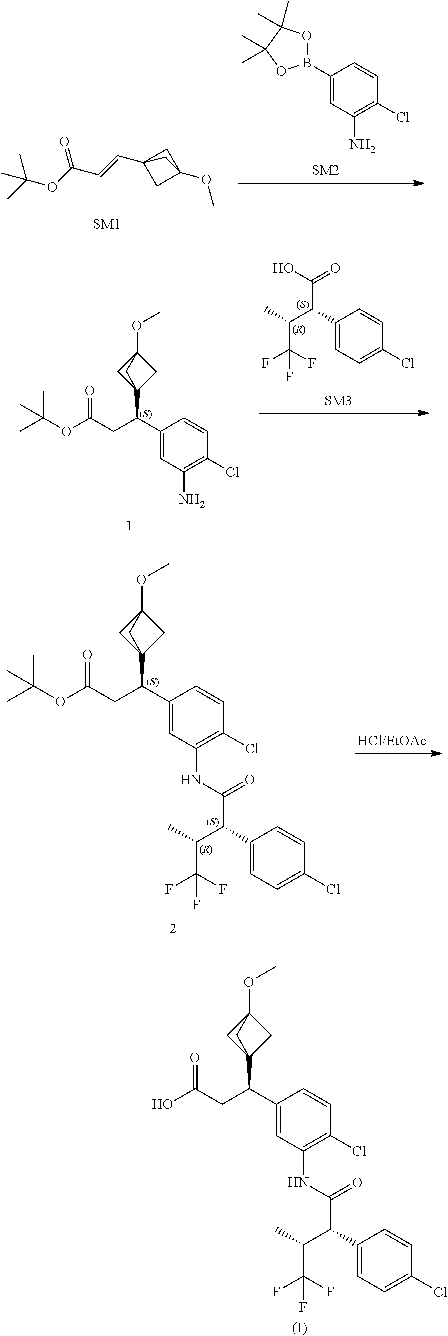

### Step 1: Synthesis of Compound SM1-7

Compound SM1-6 was dissolved in dichloromethane (120 mL). The mixture was cooled to 0° C. under nitrogen atmosphere. Oxalyl chloride (21.32 g) was added dropwise, and N,N-dimethylformamide (0.2 mL) was added dropwise. The mixture was stirred at 20° C. for 1 hour. One drop of the reaction mixture was taken, quenched with methanol, and analyzed by TLC (petroleum ether/ethyl acetate=1:1), which showed residual starting material and formation of a new spot. After the mixture was cooled to 0° C. again, additional oxalyl chloride (21.32 g) was added, and the mixture was stirred at 20° C. for 1 hour. The reaction mixture was concentrated under reduced pressure at 40° C. The residue was dissolved in dichloromethane (100 mL) and further concentrated under reduced pressure.

Methoxymethylamine hydrochloride (9.83 g) was added to dichloromethane (100 mL), followed by the addition of N,N-diisopropylethylamine (43.42 g). The mixture was cooled to 0° C. under nitrogen atmosphere, and a solution of the above compound in dichloromethane (130 mL) was added dropwise. The mixture was stirred at 20° C. for 12 hours. The reaction mixture was poured into water (100 mL). The phases were separated, and the organic phase was collected. The aqueous phase was extracted with dichloromethane (100 mL×3). The organic phases were combined and washed with saturated brine (100 mL×2), dried over anhydrous sodium sulfate, and filtered. The filtrate was concentrated under reduced pressure to remove the solvent. The crude product was purified by column chromatography (eluent: petroleum ether/ethyl acetate=1/0 to 6/1, v/v) to obtain compound SM1-7.

¹H NMR (400 MHz, CDCl₃) δ ppm 2.22 (s, 6H) 3.19 (s, 3H) 3.31 (s, 3H) 3.66 (s, 3H).

### Step 2: Synthesis of Compound SM1-8

Compound SM1-7 (11.5 g) was added to tetrahydrofuran (120 mL). The system was purged with nitrogen three times and frozen to −70° C. under nitrogen atmosphere. Diisobutylaluminum hydride (1M in toluene, 74.51 mL) was added dropwise. After the dropwise addition was completed, the mixture was stirred at −70° C. for 0.5 hours. After the reaction was completed, the reaction mixture was poured into dilute hydrochloric acid (3 mol/L, 100 mL) and stirred for 0.5 hours. The mixture was extracted with ethyl acetate (50 mL×3). The organic phases were combined, washed with saturated brine (200 mL), and dried over anhydrous sodium sulfate. The mixture was filtered, and the filtrate was concentrated under reduced pressure at 45° C. to obtain compound SM1-8.

### Step 3: Synthesis of Compound SM1

tert-Butyl diethylphosphonoacetate (15.60 g) was added to tetrahydrofuran (100 mL). The system was purged with nitrogen three times and cooled to 0° C. under nitrogen atmosphere. Potassium tert-butoxide (1M in THF, 68.01 mL) was added dropwise, and the mixture was stirred for 0.5 hours at 0° C. A solution of compound SM1-8 (7.8 g) in tetrahydrofuran (150 mL) was added dropwise at 0° C., and the mixture was stirred at 20° C. for 12 hours. After the reaction was completed, saturated aqueous ammonium chloride solution (200 mL) was added to the reaction mixture. The mixture was extracted with ethyl acetate (100 mL×2). The organic phases were combined, washed with saturated brine (200 mL), dried over anhydrous sodium sulfate, and filtered. The filtrate was concentrated under reduced pressure at 45° C. The crude product was purified by column chromatography (eluent: petroleum ether/ethyl acetate=1/0 to 97/3, v/v) to obtain compound SM1.

¹H NMR (400 MHz, CDCl₃) δ:6.95 (d, J=15.6 Hz, 1H), 5.75 (d, J=15.2 Hz, 1H), 3.31 (s, 3H), 1.99 (s, 6H), 1.49 (s, 9H).

### Step 4: Synthesis of Compound 1

Compounds SM1 (6 g) and SM2 (6.78 g) were added to tetrahydrofuran (100 mL) and isopropanol (50 mL). Potassium hydroxide (1.80 g) and 1,5-cyclooctadiene (289.39 mg) were added, and the system was purged with nitrogen three times. Under nitrogen atmosphere, the mixture was heated to 60° C., followed by the addition of a pre-prepared catalyst (the catalyst solution was prepared by adding (1,5-cyclooctadiene) rhodium(I) chloride dimer (329.75 mg) and 2,2′-bis(diphenylphosphino)-1,1′-binaphthyl (95.78 mg) into tetrahydrofuran (10 mL) with stirring for 10 minutes). The mixture was stirred at 60° C. for 12 hours. After the reaction was completed, the mixture was poured into water (200 mL) and extracted with ethyl acetate (100 mL×2). The organic phases were combined and dried over anhydrous sodium sulfate, and filtered. The filtrate was concentrated under reduced pressure at 45° C. The crude product was purified by column chromatography (eluent: petroleum ether/ethyl acetate=19/1 to 9/1, v/v) to obtain the racemic compound. The racemate was further separated by SFC (column model: DAICEL CHIRALCEL OJ (250 mm×50 mm, 10 μm); mobile phase: [0.1% ammonia in water/isopropanol] %: 15%-15%) to obtain compound 1.

¹H NMR (400 MHz, CDCl₃) δ:7.14 (d, J=8.0 Hz, 1H), 6.53 (d, J=1.6 Hz, 1H), 6.45 (dd, J=2.0, 8.2 Hz, 1H), 3.32-3.11 (m, 4H), 2.62-2.43 (m, 2H), 1.65 (s, 6H), 1.33 (s, 9H).

SFC analysis method: column type: Chiralcel OJ-3 (150 mm×4.6 mm., 3 μm); mobile phase: Phase A was CO₂, Phase B was [isopropanol (containing 0.1% isopropylamine)], gradient (B %): 10% to 50%, 5 min. The peak time of compound 1 was 1.834 min, and the retention time of its isomer was 1.960 min.

### Step 5: Synthesis of Compound 2

SM3 (2.5 g) was added to dichloromethane (25 mL), and the mixture was cooled to 0° C. under nitrogen atmosphere. Oxalyl chloride (2.38 g) was added dropwise, and N,N-dimethylformamide (0.6 mL) was added dropwise. After the dropwise addition was completed, the mixture was stirred at 20° C. for 1 hour. The reaction mixture was concentrated to dryness under reduced pressure at 40° C., dissolved in dichloromethane (10 mL), and further concentrated to dryness under reduced pressure. The resulting oily substance was dissolved in dichloromethane (30 mL) and added dropwise at 0° C. to a solution of compound 1 (3 g) and N,N-diisopropylethylamine (2.75 g, 21.31 mmol) in dichloromethane (30 mL). After the dropwise addition was completed, the mixture was stirred at 20° C. for 2 hours. After the reaction was completed, the reaction mixture was concentrated under reduced pressure. The crude product was purified by column chromatography (eluent: petroleum ether/ethyl acetate=19/1 to 9/1, v/v) to obtain compound 2.

¹H NMR (400 MHz, DMSO-d₆) δ:12.44-11.49 (m, 1H), 9.82 (s, 1H), 7.52-7.40 (m, 4H), 7.37-7.26 (m, 2H), 6.95 (dd, J=2.0, 8.4 Hz, 1H), 4.13 (d, J=10.6 Hz, 1H), 3.47-3.38 (m, 1H), 3.28-3.23 (m, 1H), 3.09 (s, 3H), 2.56 (d, J=6.4 Hz, 1H), 2.48-2.40 (m, 1H), 1.54 (q, J=9.4 Hz, 6H), 0.80 (d, J=7.0 Hz, 3H).

### Step 6: Synthesis of Compound of Formula (I)

Ethyl acetate hydrochloride (4 M, 100 mL) was added to compound 2 (4.7 g), and the mixture was stirred at 20° C. for 2 hours. After the reaction was completed, the reaction mixture was concentrated under reduced pressure. The crude product was purified by column chromatography (eluent: petroleum ether/ethyl acetate=19/1 to 17/3, v/v) to obtain the target compound (I).

### Example 2: Preparation of Crystal Form a of Compound of Formula (I)

Into a dried three-necked flask was added n-hexane (20 mL), and compound (I) (1.84 g) was added in batches. After the addition was completed, the temperature was increased to 50-55° C., and the mixture was stirred for 12 hours. The reaction mixture was filtered. The solid was collected, and vacuum dried at 45° C. to obtain crystal form A of the compound of formula (I).

¹H NMR (400 MHz, DMSO-d₆) δ ppm 12.11 (s, 1H) 9.82 (s, 1H) 7.42-7.51 (m, 4H) 7.30-7.38 (m, 2H) 6.95 (dd, J=8.4, 2.06 Hz, 1H) 4.13 (d, J=10.8 Hz, 1H) 3.37-3.43 (m, 1H) 3.27 (dd, J=8.8, 6.44 Hz, 1H) 3.10 (s, 3H) 2.54-2.62 (m, 1H) 2.43-2.48 (m, 1H) 1.54 (q, J=9.2 Hz, 6H) 0.80 (d, J=6.8 Hz, 3H).

### Example 3: Hygroscopicity Study of Crystal Form A of Compound of Formula (I)

### Experimental Materials:

SMS (Intrinsic Plus) dynamic vapor sorption analyzer.

### Experimental Methods:

Crystal form A of the compound of formula (I) (10-15 mg) was taken and placed in a DVS sample disk for testing.

### Experimental Results:

The DVS pattern of the crystal form A of the compound of formula (I) is shown in FIG. 4, where ΔW=0.13%.

### Experimental Conclusion:

Under the condition of 25° C., compared with the initial 0% RH, the sample from the crystal form A of the compound of formula (I) exhibited a moisture absorption weight gain of 0.13% at 80% RH, which is non-hygroscopic or virtually non-hygroscopic.

### Example 4: Solid Stability Test of Crystal Form A of Compound of Formula (I)

Based on the “Guideline for Stability Testing of Active Pharmaceutical Ingredients and Preparations” (Chinese Pharmacopoeia 2015 Edition, Volume IV, General Rule 9001), the stability of the crystal form A of the compound of formula (I) was investigated under high temperature (60° C., open), high humidity (room temperature/relative humidity: 92.5%, open), and strong light (5000 lx, sealed) conditions.

15 mg of the crystal form A of the compound of formula (I) was weighed and placed at the bottom of a glass sample vial, and spread into a thin layer. For samples placed under high temperature and high humidity conditions, the vial was sealed with aluminum foil punched with small holes to ensure that the samples were in full contact with the ambient air; for samples placed under strong light conditions, the vial was sealed with a threaded cap. The samples placed under different conditions were sampled and detected (XRPD) on day 5, day 10, and month 1, and the results were compared with the initial results on day 0. The test results are shown in Table 3 below:

TABLE 3
Solid stability test results of crystal
form A of compound of formula (I)

| Test conditions | Time point | Crystal form |
|---|---|---|
| — | Day 0 | Crystal form A |
| High temperature | Day 5 | Crystal form A |
| (60° C., open) | Day 10 | Crystal form A |
| | Month 1 | Crystal form A |
| High humidity | Day 5 | Crystal form A |
| (25° C./relative | Day 10 | Crystal form A |
| humidity: 92.5%, open) | Month 1 | Crystal form A |
| Strong light | Day 5 | Crystal form A |
| (5000 1x, sealed) | Day 10 | Crystal form A |
| | Month 1 | Crystal form A |
| 40° C./75% | Day 5 | Crystal form A |
| RH (open) | Day 10 | Crystal form A |
| | Month 1 | Crystal form A |
| 60° C./75% | Day 5 | Crystal form A |
| RH (open) | Day 10 | Crystal form A |
| | Month 1 | Crystal form A |

Conclusion: The crystal form A of the compound of formula (I) has good stability when subjected to high temperature, high humidity, and strong light conditions.

### Example 5: Single Crystal X-Ray Diffraction Detection Analysis of Compound of Formula (I)

### Single Crystal X-Ray Diffraction Method of the Present Disclosure

Instrument model: Single crystal X-ray diffractometer (SC-XRD) (Rigaku Oxford Diffraction XtaLAB Synergy-S).
Instrument: Rigaku Oxford Diffraction XtaLAB Synergy-S four-circle diffractometer.
Area detector: HyPix-6000HE.
Cryosystem: Oxford Cryostream 800.
Light source: Cu, λ=1.54184 Å, 50 W.
Crystal-to-CCD detector distance: d=35 mm.
Tube voltage: 50 kV.
Tube current: 1 mA.

Diffraction experiment collected 95826 diffraction points, among which 5150 diffraction points were independent (Rint=0.1139). The diffraction collection range was 2=7.056 to 133.196. The diffraction index ranges were −29≤h≤29, −13≤k≤13, −12≤1≤12. Structure solution was performed using SHELXT (Sheldrick, G. M. 2015. Acta Cryst. A71, 3-8), and structure refinement was performed using SHELXL (against F2) (Sheldrick, G. M. 2015. Acta Cryst. C71, 3-8). Among the 5150 independent diffraction points, the parameter participating in the structure refinement was 340. After refinement, R1=0.0566, wR2=0.1252. The residual electron density values were 0.36 and −0.38 eÅ−3.

Single crystal cultivation process: The sample was added with 5 mL of methanol/water solvent at a ratio of 7:5 under ultrasonic heating at 50° C., and insoluble solids were filtered out. The clear sample solution was placed in an 8 mL semi-sealed sample vial and kept in a light-proof and vibration-free environment. The sample solution was allowed to evaporate slowly at room temperature. Colorless plate-like crystals were obtained on the fourteenth day.

Conclusion: The tested crystal was colorless plate-like (0.30×0.20×0.05 mm³) and belonged to the orthorhombic crystal system, space group P21212. Unit cell parameters were: a=25.0586(5) Å, b=11.4174(2) Å, c=10.2194(2) Å, α=90°, β=90°, γ=90°, V=2923.81(10) Å3, Z=4. Calculated density Dc=1.278 g/cm³, number of electrons per unit cell F (000)=1168.0, linear absorption coefficient of the unit cell μ (Cu Kα)=2.466 mm⁻¹, diffraction experiment temperature T=150.01(10) K. Solvent subtraction treatment was applied during data refinement.

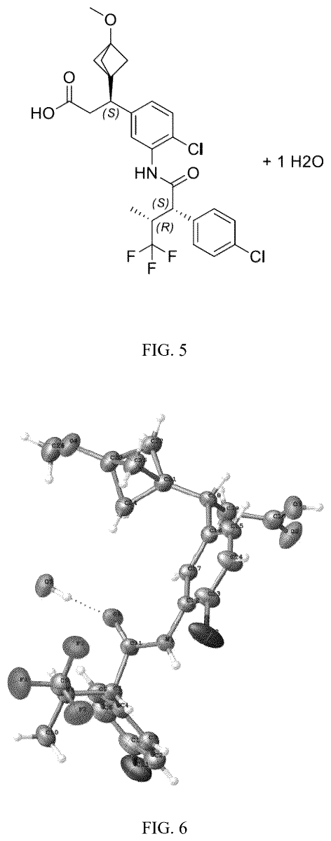

Through single-crystal X-ray diffraction analysis, it was determined that one unit cell contained one molecule of the compound of formula (I) and one solvent (water) molecule. The molecular structure diagram of the compound of formula (I) is shown in FIG. 5, and the ellipsoid plot of the molecular structure is shown in FIG. 6. Crystal structure data and parameters of the compound of formula (I) are listed in Tables 4, 5, 6, 7, and 8.

TABLE 4
Crystal data of compound of formula (I)

| | |
|---|---|
| Crystal Size | 0.30 × 0.20 × |
| | 0.05 mm³ |
| Wavelength | λ = 1.54184 Å |
| Crystal system | orthorhombic |
| Space Group | P2₁2₁2 |
| Cell Size | a = 25.0586(5) Å |
| | b = 11.4174 (2) Å |
| | c = 10.2194(2) Å |
| | α = 90° |
| | β = 90° |
| | γ = 90° |
| Cell Volume | V = 2923.81(10) Å³ |
| Cell Formula Units | Z = 4 |
| Crystal Density | D_{c} = 1.278 g/m³ |
| Crystal F(000) | 1168.0 |
| Absorption Coefficient | μ (Cu Kα) = 2.466 mm⁻¹ |
| Limiting Indices | −29 ≤ h ≤ 29 |
| | −13 ≤ k ≤ 13 |
| | −12 ≤ l ≤ 12 |
| Cell Measurement Temperature | T = 150.01 (10) K. |
| 2θ range for data collection | 7.056 to 133.196 |
| Goodness-of-fit on F\hat{ }2 | 1.055 |
| Final R indices [I > 2sigma(I)] | R1 = 0.0566, wR2 = 0.1252 |
| R indices (all data) | R1 = 0.0576, wR2 = 0.1260 |
| Largest diff. peak and hole | 0.36 and −0.38 e.Å⁻³ |
| Reflections collected/unique | 95826/5150[R₍ᵢₙₜ₎ = 0.1139] |

TABLE 5
Atomic coordinates (×10⁴) and equivalent isotropic
displacement parameters (Å² × 10³)
of crystals of compound of formula (I)

| | x | y | z | U(eq) |
|---|---|---|---|---|
| Cl2 | 8995.9(5) | 3606.2(19) | 6554.5(12) | 83.9(6) |
| Cl1 | 5192.2(5) | 3040.2(17) | 3117.1(16) | 82.7(5) |
| F3 | 7935.4(11) | 6610(3) | 6714(3) | 65.3(8) |
| F2 | 7514.6(13) | 5809(3) | 8289(3) | 68.7(8) |
| O1 | 7640.7(4) | 5815(3) | 3929(3) | 48.5(8) |
| O5 | 7231.5(14) | 7800(3) | 2791(3) | 48.2(8) |
| F1 | 7367.4(15) | 7607(3) | 7795(4) | 80.4(10) |
| N1 | 8031.4(13) | 4310(3) | 5023(3) | 35.8(7) |
| O3 | 9259.4(13) | 2546(3) | 166(4) | 55.7(9) |
| O4 | 8669.9(15) | 8872(3) | 309(4) | 59.5(9) |
| O2 | 8481.5(16) | 2124(3) | −755(4) | 63.5(10) |
| C17 | 8526.2(16) | 4514(3) | 2959(4) | 33.8(8) |
| C12 | 8510.4(16) | 4257(4) | 4284(4) | 35.0(8) |
| C16 | 8997.4(16) | 4454(3) | 2234(4) | 35.2(8) |
| C11 | 7632.9(16) | 5093(4) | 4807(4) | 36.9(9) |
| C4 | 6679.5(17) | 4474(4) | 5003(4) | 39.7(9) |
| C18 | 9010.3(18) | 4809(4) | 810(4) | 37.2(9) |
| C7 | 7156.7(16) | 4976(4) | 5737(4) | 38.3(9) |
| C20 | 8780(2) | 2821(4) | −243(4) | 45.7(10) |
| C15 | 9457.5(17) | 4075(4) | 2871(5) | 45.0(10) |
| C21 | 8892.1(18) | 6089(4) | 655(4) | 40.4(10) |
| C8 | 7021.4(18) | 6171(4) | 6368(4) | 42.7(10) |
| C9 | 7455(2) | 6541(4) | 7278(5) | 50.8(11) |
| C23 | 8754(2) | 7668(4) | 430(5) | 49.0(11) |
| C24 | 8363.1(18) | 6755(4) | 993(5) | 43.2(10) |
| C25 | 9196(2) | 7146(4) | 1277(5) | 52.5(12) |
| C19 | 8632.7(19) | 4082(4) | −48 (4) | 42.6(10) |
| C3 | 6498.6(18) | 3369(4) | 5307(5) | 48.4(11) |
| C13 | 8982.5(18) | 3912(5) | 4891(4) | 49.7(11) |
| C22 | 8900(2) | 6784(4) | −661(5) | 53.2(12) |
| C14 | 9451.5(18) | 3801(5) | 4187(5) | 52.5(12) |
| C2 | 6048(2) | 2906(5) | 4708(6) | 58.6(13) |
| C5 | 6416.1(19) | 5111(5) | 4036(5) | 50.2(11) |
| C1 | 5783.3(19) | 3586(5) | 3799(5) | 58.8(14) |
| C26 | 8201(2) | 9152(4) | −434(6) | 63.5(14) |

TABLE 6
Bond length (Å) of compound of formula (I)

| | Bond length/Å | | Bond length/Å |
|---|---|---|---|
| Cl(2)—C(13) | 1.736(5) | C(4)—C(5) | 1.393(6) |
| Cl(1)—C(1) | 1.751(5) | C(18)—C(21) | 1.499(6) |
| F(3)—C(9) | 1.337(6) | C(18)—C(19) | 1.534(6) |
| F(2)—C(9) | 1.337(6) | C(7)—C(8) | 1.547(6) |
| O(1)—C(11) | 1.218(5) | C(20)—C(19) | 1.499(6) |
| F(1)—C(9) | 1.345(6) | C(15)—C(14) | 1.380(7) |
| N(1)—C(12) | 1.419(5) | C(21)—C(24) | 1.567(6) |
| N(1)—C(11) | 1.359(6) | C(21)—C(25) | 1.562(6) |
| O(3)—C(20) | 1.311(6) | C(21)—C(22) | 1.562(6) |
| O(4)—C(23) | 1.397 (5) | C(8)—C(9) | 1.492(7) |
| O(4)—C(26) | 1.435 (7) | C(8)—C(10) | 1.531(6) |
| O(2)—C(20) | 1.410(7) | C(23)—C(24) | 1.541(6) |
| C(17)—C(12) | 1.387(6) | C(23)—C(25) | 1.527(7) |
| C(17)—C(16) | 1.396(6) | C(23)—C(22) | 1.547(7) |
| C(12)—C(13) | 1.393(6) | C(3)—C(2) | 1.390(7) |
| C(16)—C(18) | 1.511(6) | C(13)—C(14) | 1.384(6) |
| C(16)—C(15) | 1.393(6) | C(2)—C(1) | 1.380(8) |
| C(11)—C(7) | 1.531(6) | C(5)—C(6) | 1.394(7) |
| C(4)—C(7) | 1.523(6) | C(1)—C(6) | 1.353(8) |
| C(4)—C(3) | 1.377(6) | | |

TABLE 7
Bond angle (°) of compound of formula (I)

| | Bond | | Bond |
|---|---|---|---|
| | angle/° | | angle/° |
| C(11)—N(1)—C(12) | 124.3(3) | C(9)—C(8)—C(7) | 100.5(4) |
| C(23)—O(4)—C(26) | 112.9(4) | C(9)—C(8)—C(10) | 109.6(4) |
| C(12)—C(17)—C(16) | 122.2(4) | C(10)—C(8)—C(7) | 112.6(4) |
| C(17)—C(12)—N(1) | 122.3(4) | F(3)—C(9)—F(2) | 105.7(4) |
| C(17)—C(12)—C(13) | 118.1(4) | F(3)—C(9)—F(1) | 105.3(4) |
| C(13)—C(12)—N(1) | 119.6(4) | F(3)—C(9)—C(8) | 113.8(4) |
| C(17)—C(16)—C(18) | 121.1(4) | F(2)—C(9)—F(1) | 106.3(4) |
| C(15)—C(16)—C(17) | 117.9(4) | F(2)—C(9)—C(8) | 112.7(4) |
| C(15)—C(16)—C(18) | 121.1(4) | F(1)—C(9)—C(8) | 112.4(4) |
| O(1)—C(11)—N(1) | 123.5(4) | O(4)—C(23)—C(24) | 127.1(4) |
| O(1)—C(11)—C(7) | 121.9(4) | O(4)—C(23)—C(25) | 122.9(4) |
| N(1)—C(11)—C(7) | 114.5(3) | O(4)—C(23)—C(22) | 127.9(4) |
| C(3)—C(4)—C(7) | 119.5(4) | C(24)—C(23)—C(22) | 88.8(3) |
| C(3)—C(4)—C(5) | 118.8(4) | C(25)—C(23)—C(24) | 89.2(4) |
| C(5)—O(4)—C(7) | 121.7(4) | C(25)—C(23)—C(22) | 88.9(4) |
| C(16)—C(18)—C(19) | 113.1(3) | C(23)—C(24)—C(21) | 73.1(3) |
| C(21)—C(18)—C(16) | 111.0(3) | C(23)—C(25)—C(21) | 73.6(3) |
| C(21)—C(18)—C(19) | 110.2(4) | C(20)—C(19)—C(18) | 116.3(4) |
| C(11)—C(7)—C(8) | 110.6(4) | C(4)—C(3)—C(2) | 121.2(5) |
| C(4)—C(7)—C(11) | 109.8(3) | C(12)—C(13)—Cl(2) | 120.6(3) |
| C(4)—C(7)—C(8) | 111.4(3) | C(14)—C(13)—Cl(2) | 118.3(3) |
| O(3)—C(20)—C(19) | 114.4(4) | C(14)—C(13)—C(12) | 121.0(4) |
| O(2)—C(20)—O(3) | 123.1(5) | C(23)—C(22)—C(21) | 73.0(3) |
| O(2)—C(20)—C(19) | 122.5(5) | C(15)—C(14)—C(13) | 119.7(4) |
| C(14)—C(15)—C(16) | 121.1(4) | C(1)—C(2)—C(3) | 118.2(5) |
| C(18)—C(21)—C(24) | 128.1(4) | C(4)—C(5)—C(6) | 120.4(5) |
| C(18)—C(21)—C(25) | 127.9(4) | C(2)—C(1)—Cl(1) | 118.3(4) |
| C(18)—C(21)—C(22) | 125.7(4) | C(6)—C(1)—Cl(1) | 119.4(5) |
| C(25)—C(21)—C(24) | 87.0(3) | C(6)—C(1)—C(2) | 122.3(5) |
| C(22)—C(21)—C(24) | 87.4(3) | C(1)—C(6)—C(5) | 119.0(5) |
| C(22)—C(21)—C(25) | 87.2(3) | | |

TABLE 8
Torsion angle (°) of compound of formula (I)

| | Torsion | | Torsion |
|---|---|---|---|
| | angle/° | | angle/° |
| Cl(2)—C(13)—C(14)—C(15) | −177.9(4) | C(18)—C(21)—C(25)—C(23) | 178.5(5) |
| Cl(1)—C(1)—C(6)—C(5) | 176.7(4) | C(28)—C(21)—C(22)—C(23) | −179.5(5) |
| O(1)—C(11)—C(7)—C(4) | 69.8(5) | C(7)—C(4)—C(3)—C(2) | 176.6(4) |
| O(1)—C(11)—C(7)—C(8) | −53.6(6) | C(7)—C(4)—C(5)—C(6) | −176.0(4) |
| N(1)—C(12)—C(13)—Cl(2) | −2.7(6) | C(7)—C(8)—C(9)—F(3) | 57.0(5) |
| N(1)—C(12)—C(13)—C(14) | 177.1(5) | C(7)—C(8)—C(9)—F(2) | −63.3(5) |
| N(1)—C(11)—C(7)—C(4) | −108.3(4) | C(7)—C(8)—C(9)—F(1) | 176.6 (4) |
| N(1)—C(11)—C(7)—C(8) | 128.3(4) | C(15)—C(16)—C(18)—C(21) | −114.4(4) |
| O(3)—C(20)—C(19)—C(18) | −10.8(6) | C(15)—C(16)—C(18)—C(19) | 121.1(5) |
| O(4)—C(23)—C(24)—C(21) | −176.5(5) | C(21)—C(18)—C(19)—C(20) | 166.3(4) |
| O(4)—C(23)—C(25)—C(21) | 179.6(5) | C(24)—C(21)—C(25)—C(23) | −43.6(3) |
| O(4)—C(23)—C(22)—C(21) | 176.9(5) | C(24)—C(21)—C(22)—C(23) | 43.8(3) |
| O(2)—C(20)—C(19)—C(18) | 170.3(4) | C(24)—C(23)—C(25)—C(21) | 44.5(3) |
| C(17)—C(12)—C(13)—C(12) | 178.7(4) | C(24)—C(23)—C(22)—C(21) | −44.7(3) |
| C(17)—C(12)—C(13)—C(14) | −1.6(7) | C(25)—C(21)—C(24)—C(23) | 43.3(3) |
| C(17)—C(16)—C(18)—C(21) | 64.5(5) | C(25)—C(21)—C(22)—C(23) | −43.3(3) |
| C(17)—C(16)—C(18)—C(19) | −60.0(5) | C(25)—C(23)—C(24)—C(21) | −44.5(3) |
| C(17)—C(16)—C(15)—C(14) | −1.9(7) | C(25)—C(23)—C(22)—C(21) | 44.5(3) |
| C(12)—N(1)—C(11)—O(1) | 2.0(7) | C(19)—C(18)—C(21)—C(24) | 62.0(6) |
| C(12)—N(1)—C(11)—C(7) | −179.9(4) | C(19)—C(18)—C(21)—C(25) | −176.3(4) |
| C(12)—C(17)—C(16)—C(18) | −176.3(4) | C(19)—C(18)—C(21)—C(22) | −57.4(6) |
| C(12)—C(17)—C(16)—C(15) | 2.7(6) | C(3)—C(4)—C(7)—C(11) | 112.2(4) |
| C(12)—C(13)—C(14)—C(15) | 2.3(8) | C(3)—C(4)—C(7)—C(8) | −124.8(4) |
| C(16)—C(17)—C(12)—N(1) | −179.6(4) | C(3)—C(4)—C(5)—C(6) | 3.4(7) |
| C(16)—C(17)—C(12)—C(13) | −1.0(6) | C(3)—C(2)—C(1)—C1(1) | −176.1(4) |
| C(16)—C(18)—C(21)—C(24) | −64.1(6) | C(3)—C(2)—C(1)—C(6) | 2.8(8) |
| C(16)—C(18)—C(21)—C(25) | 57.6(6) | C(22)—C(21)—C(24)—C(23) | −44.0(3) |
| C(16)—C(18)—C(21)—C(22) | 176.5(4) | C(22)—C(21)—C(25)—C(23) | 43.9(3) |
| C(16)—C(18)—C(19)—C(20) | −68.8(5) | C(22)—C(23)—C(24)—C(21) | 44.5(3) |
| C(16)—C(15)—C(14)—C(13) | −0.5(8) | C(22)—C(23)—C(25)—C(21) | −44.3(3) |
| C(11)—N(1)—C(12)—C(17) | −34.1(6) | C(2)—C(1)—C(6)—C(5) | −2.2(8) |
| C(11)—N(1)—C(12)—C(13) | 147.3(5) | C(5)—C(4)—C(7)—C(11) | −68.4(5) |
| C(11)—C(7)—C(8)—C(9) | −67.9(5) | C(5)—C(4)—C(7)—C(8) | 54.5(5) |
| C(11)—C(7)—C(8)—C(10) | 169.2(4) | C(5)—C(4)—C(3)—C(2) | −2.8(7) |
| C(4)—C(7)—C(8)—C(9) | 169.6(4) | C(26)—O(4)—C(23)—C(24) | −56.4(7) |
| C(4)—C(7)—C(8)—C(10) | 46.7(5) | C(26)—O(4)—C(23)—C(25) | −174.2(5) |
| C(4)—C(3)—C(2)—C(1) | −0.2(7) | C(26)—O(4)—C(23)—C(22) | 67.4(7) |
| C(4)—C(5)—C(6)—C(1) | −0.9(8) | C(10)—C(8)—C(9)—F(3) | −178.4(4) |
| C(18)—C(16)—C(15)—C(14) | 177.0(4) | C(10)—C(8)—C(9)—F(2) | 61.3(5) |
| C(18)—C(21)—C(24)—C(23) | −179.0(5) | C(10)—C(8)—C(9)—F(1) | −58.8(5) |

### Bioassay

### Test Example 1: In Vitro Activity Assay

### I. CGMP Expression Test Based on InCap Cells

### 1. Experimental Steps

### 1) Solution Preparation

### 10% BSA (Bovine Serum Albumin)

10 g of BSA was dissolved in 100 mL of double-distilled water (ddH₂O) to obtain 10% BSA.

### 10 mM ODQ Stock Solution

1 mg of ODQ powder was weighed and dissolved in 534 μL of DMSO to prepare 10 mM ODQ solution. The solution was aliquoted and stored in a −20° C. refrigerator.

### Washing Buffer (50 mL)

| | Final |
|---|---|
| Volume | concentration |
| 49 mL of Earls balanced | 1x |
| salt solution (EBSS) | |
| 500 μL of 1M hydroxyethyl piperazine | 10 mM |
| ethanesulfonic acid (HEPES) | |
| 250 μL of 10% BSA stabilizer | 0.05% |
| 250 μL of 1M MgCl₂ | 5 mM |

### Assay Buffer (50 mL)

| | Final |
|---|---|
| Volume | concentration |
| 48.95 mL of Earls balanced | 1x |
| salt solution (EBSS) | |
| 500 μL of 1M hydroxyethyl piperazine | 10 mM |
| ethanesulfonic acid (HEPES) | |
| 250 μL 10% BSA | 0.05% |
| 50 μL of 500 mmol/L | 0.5 mM |
| isobutylmethylxanthine (IBMX) | |
| 250 μL of 1M MgCl₂ | 5 mM |

### Detection Buffer

a) 50 μL of cGMP-D2 (D2-labeled cyclic guanosine monophosphate) was added to 1 mL of lysis buffer and mixed thoroughly.
b) 50 μL of anti-cGMP cryptate (Eu³⁺ cryptate-labeled anti-cyclic guanosine monophosphate antibody) was added to 1 mL of lysis buffer and mixed thoroughly.

### 2) Compound Dilution

(1) The compound was diluted to a concentration of 10 mM using DMSO.
(2) A serial dilution of the compound was performed, resulting in 10 different concentration levels of each compound. 100 nL of each diluted compound was added to a 96-well plate.

### 3) Preparation of LNCap Cells

(1) LNCap culture medium composition: RPMI1640 supplemented with 10% fetal bovine serum (FBS) and 1% double antibody.
(2) The phosphate-buffered saline (PBS), trypsin, and culture medium for use in cell passage were preheated in a 37° C. water bath.
(3) Cells were removed from a 37° C., 5% CO₂ incubator, and the old culture medium was removed from the culture flask using a pipette.
(4) 5 mL of PBS was pipetted into the flask for rinsing the cells, and then the liquid was discarded.
(5) 3 mL of trypsin was pipetted into the flask. After shaking, the liquid was discarded, and the flask was returned to the incubator.
(6) Approximately 2 minutes later, the flask was retrieved, and cell detachment was observed. 9 mL of culture medium was pipetted into the culture flask and mixed by repetitive pipetting. The cell suspension was then transferred to a 50 mL centrifuge tube.
(7) 0.7 mL of cell suspension was pipetted into a counting chamber, and cell counting was performed using the ViCell XR. The remaining cells were centrifuged at 1000 rpm for 5 minutes, and the supernatant was discarded.
(8) 10 mL of washing buffer was added to wash the cells, followed by another centrifugation at 1000 rpm for 5 minutes, after which the supernatant was discarded.
(9) Assay buffer was added, and the cell concentration was adjusted to 3×10⁶/mL.

### 4) Preparation and Addition of ODQ Solution

(1) 10 mM ODQ stock solution was diluted at a ratio of 1:1000 and added to the cell solution.
(2) After thorough mixing, the solution was added to a microplate at 10 μL per well.
   5) Preparation of cGMP Standard Curve
(1) 1 mM cGMP stock solution was diluted to 10 μM using assay buffer. 11 concentration gradients were created through 4-fold serial dilutions.
(2) The diluted cGMP was added to the microplate at 10 μL per well.

### 6) Addition of Assay Reagents and Plate Reading

(1) cGMP-D2 was transferred to a 384-well plate at 5 μL per well. Anti-cGMP cryptate was transferred to a 96-well plate at 5 μL per well. Centrifugation was performed at 1500 rpm for 1 minute.
(2) Incubation was carried out at room temperature for 1 hour.
(3) The plates were read using envision at 665/615 wavelengths.

### 7) Data Analysis

(1) cGMP standard curve: A standard curve was generated using GraphPad Prism, based on the ratio of cGMP concentration to the 665/615 wavelength values.
(2) Conversion of HTRF (homogeneous time-resolved fluorescence) ratio (665/615) to cGMP concentration: In GraphPad Prism, the HTRF ratio (665/615) was copied to the ratio column of the cGMP standard curve. The analysis “Log inhibitor vs response-variable slope” was run, and the “interpolate” option was selected to convert the HTRF ratio (665/615) into cGMP concentration.
(3) Compound activation curve: A curve was generated using the “Log agonist vs response-variable slope” analysis method in GraphPad Prism, based on the converted cGMP concentration and compound concentration.

TABLE 9
MEC values of the compounds of the present
disclosure for sGC stimulatory activity

| Compound | MEC (nM) | EC50 (nM) |
|---|---|---|
| Crystal form A | 6.93 | 21.19 |
| of compound of | | |
| formula (I) | | |

MEC: Minimum effective concentration required to stimulate cGMP production in LNCap cells (greater than three times the baseline value).
Experimental conclusion: The compounds of the present disclosure are effective in stimulating sGC, thereby increasing cGMP levels.

### Test Example 2: In Vitro Hepatocyte Metabolic Stability Study

1. Experimental objective: The aim of the study was to assess the stability of the compounds in hepatocytes of different species.
2. Experimental steps:

A variety of 96-well precipitation plates were prepared and labeled as T0, T15, T30, T60, T90, T0-MC, T90-MC, and blank matrix, respectively. Revival medium and incubation medium were taken in advance and pre-warmed in a 37° C. water bath. Hepatocytes of different species, stored in a liquid nitrogen tank, were taken and immediately immersed in a 37° C. water bath for approximately 90 seconds. Once partially thawed, cells were transferred to centrifuge tubes containing 40 mL of revival medium, and gently inverted to resuspend the cells in the revival medium. At room temperature, cells were centrifuged at 100×g for 5 minutes. The supernatant was discarded, and the hepatocytes were resuspended in an appropriate volume of incubation medium. Viability was calculated using trypan blue staining. 198 μL of hepatocyte suspension (0.51×10⁶ cells/mL) was added to pre-warmed incubation plates. For the control group, 198 μL of hepatocyte-free incubation medium was added to T0-MC and T120-MC plates. All plates were pre-incubated for 10 minutes in a 37° C. incubator. 2 μL of test and control compound working solutions were then added, mixed thoroughly, and the incubation plates were immediately placed in a shaker within the incubator. The timer was initiated to start the reaction. Two duplicate samples were prepared at each time point for each compound. Incubation conditions were set at 37° C., saturated humidity, and 5% CO₂. In the test system, the test compound had a final concentration of 1 μM, the control compound had a final concentration of 3 μM, the hepatocyte had a final concentration of 0.5×10⁶ cells/mL, and the total organic solvent had a final concentration of 0.96%, of which DMSO had a final concentration of 0.1%. At the end of the incubation at the corresponding time point, the incubation plate was removed, and 25 μL of the compound and control compound-cell mixture was transferred to a sample plate containing 125 μL of stop solution (acetonitrile solution containing 200 ng/mL of tolbutamide and Labetalol). For the blank sample plate, 25 μL of hepatocyte-free incubation medium was directly added. All sample plates were sealed and shaken on the shaker at 600 rpm for 10 minutes, and then centrifuged at 3220×g for 20 minutes. The supernatant of test and control samples was diluted with ultrapure water at a ratio of 1:3. All samples were mixed well and analyzed using LC/MS/MS methods.

The experimental results are shown in Table 10.

TABLE 10
Stability of the compounds of the present disclosure
in hepatocytes of different species

| Species | Test item | Compound of formula (I) |
|---|---|---|
| Mouse | T_{1/2}-min | 85.2 |
| | CL_{int (liver)} | 193.3 |
| | (mL/min/kg) | |
| Rat | T_{1/2}-min | 78.2 |
| | CL_{int (liver)} | 82.9 |
| | (mL/min/kg) | |
| Human | T_{1/2}-min | 154.7 |
| | CL_{int (liver)} | 24.9 |
| | (mL/min/kg) | |

T_{1/2}: half-life; CL_{int (liver)}: hepatic clearance.

Experimental conclusion: The compounds of the present disclosure exhibit good stability in human-derived hepatocytes, with moderate hepatic clearance and half-life.

### Experimental Example 3: In Vivo PK Study of Crystal Form a of Compound of Formula (I) in SD Male Rats

Experimental purpose: To test the PK properties of the compound in SD rats in vivo

Experimental materials: Two male Sprague Dawley rats, 7-8 weeks old (Beijing Vital River Laboratory Animal Technology Co., Ltd.).

Experimental procedures: The PK characteristics of the compound were assessed following oral gavage administration in rodents using a standard protocol. The test compound was formulated as a homogeneous suspension (vehicle for the oral gavage formulation: 0.5% MC+0.2% Tween 80/H₂O) and crystal form A of the compound of formula (I) was administered at a concentration of 1 mg/mL for a single oral gavage dose. Animal body weights were measured prior to dosing (initial weight range: 230-240 g), and the dosing volume was calculated based on body weight (10 mg/kg). Oral gavage was performed accordingly. Approximately 0.2 mL of a whole blood sample was collected at the specified time by jugular venipuncture (or other appropriate blood collection site), and the actual blood collection time was recorded in the experimental record, with acceptable deviations of +1 min for time points within 1 h post-dosing and +5% for other time points. Blood samples were centrifuged at 3200×g for 10 min at 4° C. to separate the supernatant and obtain plasma. The plasma was transferred to pre-chilled, labeled commercial K₂-EDTA tubes, rapidly frozen on dry ice, and stored at −70±10° C./−60° C. or lower in an ultra-low temperature freezer. Plasma drug concentration was quantitatively analyzed by LC-MS/MS analysis method, and the plasma drug concentration data of the metabolite of the compound of the present disclosure were processed by using the pharmacokinetic software of WinNonlin Version 6.3 or above (Pharsight) in a non-compartmental model. Relevant pharmacokinetic parameters such as peak concentration (Cₘₐₓ), half-life (T½), area under the curve (AUC), and time to peak (Tₘₐₓ) were calculated using the linear-log trapezoidal method. Experimental results are shown in Table 11.

TABLE 11
In vivo pharmacokinetic experimental results
of crystal form A of compound of formula (I)

| | | Crystal form A of |
|---|---|---|
| Test sample | | compound of formula (I) |
| po (10 mg/kg) | Cₘₐₓ (nmol/L) | 13423 |
| | Tₘₐₓ (h) | 0.38 |
| | AUC (h*nmol/L) | 87664 |
| | T_{1/2} (h) | 3.43 |

Experimental conclusion: The crystal form A of the compound of formula (I) has excellent pharmacokinetic properties.

1. A crystal form A of a compound of formula (I), 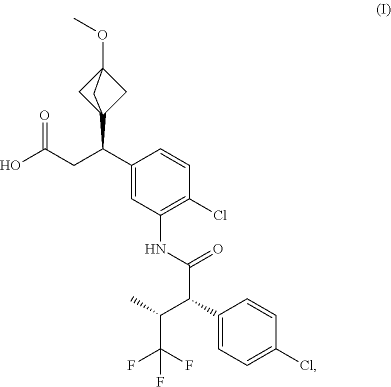 [SMILES: COC12CC([C@H](CC(=O)O)c3ccc(Cl)c(NC(=O)[C@H](c4ccc(Cl)cc4)[C@@H](C)C(F)(F)F)c3)(C1)C2]
wherein the crystal form A of the compound of formula (I) has an X-ray powder diffraction pattern comprising characteristic diffraction peaks at the following 2θ angles: 15.540±0.200°, 16.100±0.200°, and 17.601±0.200°.

2. The crystal form A of the compound of formula (I) according to claim 1, wherein the X-ray powder diffraction pattern for the crystal form A of the compound of formula (I) comprises characteristic diffraction peaks at the following 2θ angles: 11.041±0.200°, 14.381±0.200°, 15.540±0.200°, 16.100±0.200°, 17.60±10.200°, 18.281±0.200°, 18.799±0.200°, and 22.903±0.200°.

3. The crystal form A of the compound of formula (I) according to claim 2, wherein the X-ray powder diffraction pattern for the crystal form A of the compound of formula (I) comprises characteristic diffraction peaks at the following 2θ angles: 3.441±0.200°, 8.113±0.200°, 8.799±0.200°, 11.041±0.200°, 14.381±0.200°, 15.540±0.200°, 16.100±0.200°, 17.601±0.200°, 18.281±0.200°, 18.799±0.200°, 22.903±0.200°, and 23.682±0.200°.

4. The crystal form A of the compound of formula (I) according to claim 3, wherein the X-ray powder diffraction pattern for the crystal form A of the compound of formula (I) comprises characteristic diffraction peaks at the following 2θ angles: 3.441±0.200°, 7.198±0.200°, 8.113±0.200°, 8.799±0.200°, 11.041±0.200°, 13.863±0.200°, 14.381±0.200°, 15.540±0.200°, 16.100±0.200°, 17.601±0.200°, 18.281±0.200°, 18.799±0.200°, 19.523±0.200°, 22.903±0.200°, 23.682±0.200°, and 24.940±0.200°.

5. The crystal form A of the compound of formula (I) according to claim 4, wherein the X-ray powder diffraction pattern for the crystal form A of the compound of formula (I) comprises one or more of characteristic diffraction peaks at the following 2θ angles: 9.914±0.200°, 11.880±0.200°, 17.063±0.200°, 20.333±0.200°, 21.164±0.200°, 21.761±0.200°, 22.176±0.200°, 25.823±0.200°, 26.539±0.200°, 28.124±0.200°, 28.756±0.200°, 30.261±0.200°, 31.477±0.200° 32.582±0.200° 36.303±0.200° 38.141±0.200°, and 38.742±0.200°.

6. (canceled)

7. The crystal form A of the compound of formula (I) according to claim 1, wherein the crystal form A of the compound of formula (I) has a differential scanning calorimetry curve comprising an endothermic peak with an onset at 164.69° C.±5° C.

8. The crystal form A of the compound of formula (I) according to claim 7, wherein the crystal form A of the compound of formula (I) has a DSC pattern as shown in FIG. 2.

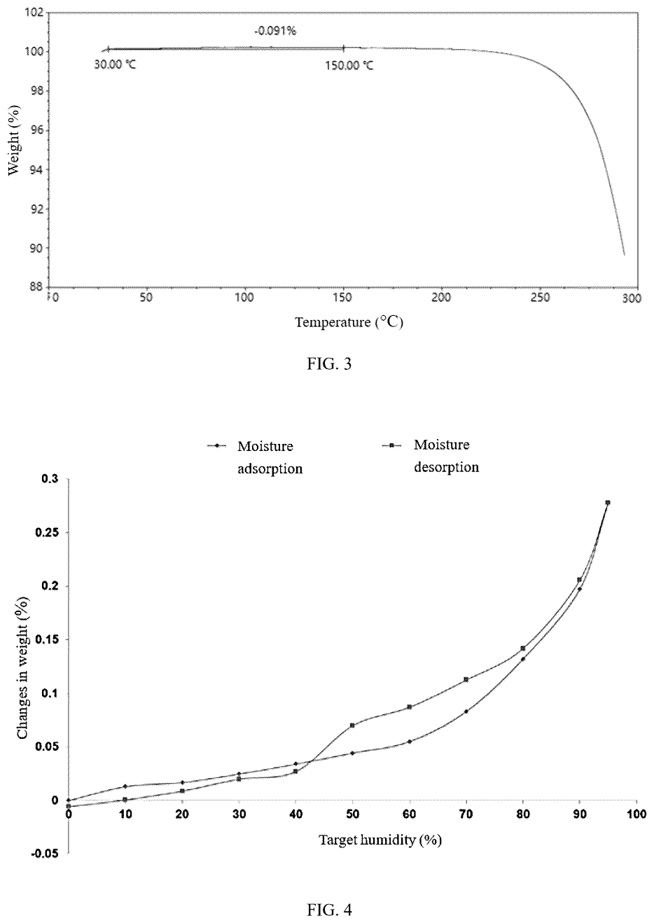

9. The crystal form A of the compound of formula (I) according to claim 1, wherein the crystal form A of the compound of formula (I) has a TGA pattern as shown in FIG. 3.

10. (canceled)

11. The crystal form A of the compound of formula (I) according to claim 5, wherein the X-ray powder diffraction pattern for the crystal form A of the compound of formula (I) comprises characteristic diffraction peaks at the following 2θ angles: 3.441±0.200°, 7.198±0.200°, 8.113±0.200°, 8.799±0.200°, 9.914±0.200°, 11.041±0.200°, 11.880±0.200°, 13.863±0.200°, 14.381±0.200°, 15.540±0.200°, 16.100±0.200°, 17.063±0.200°, 17.601±0.200°, 18.281±0.200°, 18.799±0.200°, 19.523±0.200°, 20.333±0.200°, 21.164±0.200°, 21.761±0.200°, 22.176±0.200°, 22.903±0.200°, 23.682±0.200°, 24.94±010.200°, 25,823±0.200°, 26.539±0.200° 28.124±0.200°, 28.756±0.200°, 30.261±0.200°, 31.477±0.200°, 32.582±0.200°, 36.303±0.200°, 38.141±0.200°, and 38.742±0.200°.

12. A method for treating chronic kidney disease in a subject in need thereof, comprising administering a therapeutically effective amount of the crystal form A of the compound of formula (I) according to claim 1 to the subject.

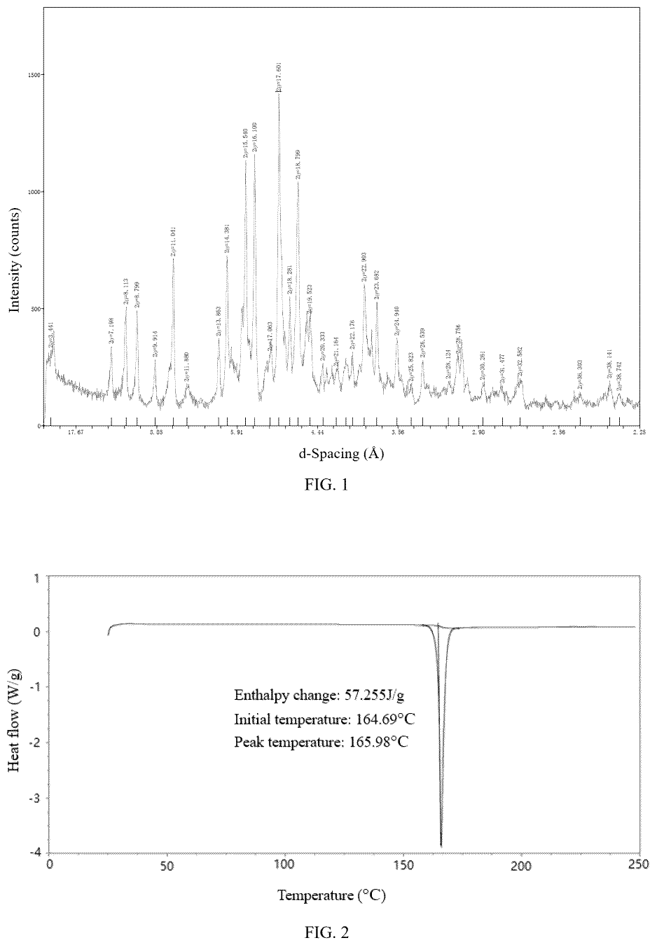

13. A crystal form A of a compound of formula (I), 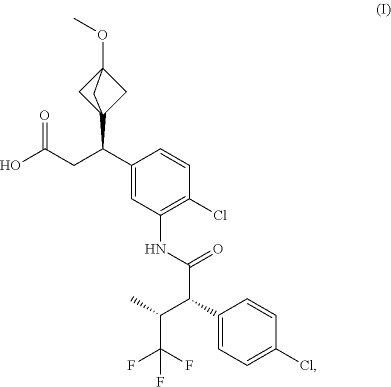 [SMILES: COC12CC([C@H](CC(=O)O)c3ccc(Cl)c(NC(=O)[C@H](c4ccc(Cl)cc4)[C@@H](C)C(F)(F)F)c3)(C1)C2]
wherein the crystal form A of the compound of formula (I) has an XRPD pattern as shown in FIG. 1.

14. The crystal form A of the compound of formula (I) according to claim 13, wherein the crystal form A of the compound of formula (I) has a differential scanning calorimetry curve comprising an endothermic peak with an onset at 164.69° C.±5° C.

15. The crystal form A of the compound of formula (I) according to claim 14, wherein the crystal form A of the compound of formula (I) has a DSC pattern as shown in FIG. 2.

16. The crystal form A of the compound of formula (I) according to claim 13, wherein the crystal form A of the compound of formula (I) has a TGA pattern as shown in FIG. 3.

17. A method for treating chronic kidney disease in a subject in need thereof, comprising administering a therapeutically effective amount of the crystal form A of the compound of formula (I) according to claim 13 to the subject.
